(19) United States
(12) Patent Application Publication
(10) Pub. No.: US 2021/0198807 A1
(43) Pub. Date: Jul. 1, 2021

(54) METHODS AND SYSTEMS FOR PRODUCTION OF DNA LIBRARIES DIRECTLY FROM A STOOL SAMPLE FOR 16S METAGENOMICS NEXT GENERATION SEQUENCING

(71) Applicant: URIT Medical Electronic Co., Ltd., Guilin (CN)

(72) Inventors: Tom Cheng XU, Castro Valley, CA (US); Chengfeng JIANG, Guilin (CN); Jinlan XU, Guilin (CN); Chunyan LIAO, Guilin (CN)

(73) Assignee: URIT Medical Electronic Co., Ltd., Guilin (CN)

(21) Appl. No.: 16/821,451

(22) Filed: Mar. 17, 2020

Related U.S. Application Data
(60) Provisional application No. 62/955,711, filed on Dec. 31, 2019.

(51) Int. Cl. C40B 50/18; C12Q 1/6806; C12Q 1/686; C12N 15/10

(52) U.S. Cl. CPC C40B 50/18; C12N 15/1065; C12Q 1/686; C12Q 1/6806

(57) ABSTRACT

Disclosed are methods for preparing a DNA library directly from a stool sample. The method comprises applying the stool sample directly to a buffer, heating and cooling the buffer, separating a supernatant within the buffer from a precipitate using centrifugation, and transferring the supernatant into a first reaction vessel containing a first reagent mixture to yield a first reaction mixture. The method also comprises subjecting the first reaction mixture to a first PCR protocol, purifying amplicons within the first reaction vessel through a first purification procedure to yield a purified target amplicon solution, transferring the purified target amplicon solution to a second reaction vessel comprising a second reagent mixture to yield a second reaction mixture, and subjecting the second reaction mixture to a second PCR protocol. The method further comprises purifying index-tagged amplicons within the second reaction vessel through a second purification procedure to yield the DNA library.

### CROSS-REFERENCE TO RELATED APPLICATION

This application claims the benefit of priority to U.S. Provisional Patent Application No. 62/955,711, filed on Dec. 31, 2019, the content of which is hereby incorporated by reference in its entirety.

### TECHNICAL FIELD

The present disclosure relates generally to the field of sample preparation for genetic sequencing; more specifically, to methods, compositions, and kits for the production of deoxyribonucleic acid (DNA) libraries directly from a stool sample for next generation sequencing.

### BACKGROUND

Metagenomics is a molecular tool used to analyze DNA sequences obtained from environmental samples in order to study the community of microorganisms present. The human intestinal tract has a variety of microbial communities that play an important role in the health of the human host. Advances in high-throughput DNA next generation sequencing (NGS) technology have enabled researchers to more quickly and efficiently reveal changes in the composition and function of gut microbes, which are often associated with various diseases or disease states, including cancer, AIDS and other illnesses. In addition, metagenomic analysis provides a better understanding of normal gut microbiome and its relationship to various exogenous and endogenous host factors. Recent studies have shown that microbiome distribution or the distribution of bacterial species within a person's gut can be used not only for early diagnosis and prognosis of disease, but also for individualized treatment options in everyday medical practice.

The use of DNA NGS technology to analyze microbial populations is generally divided into the following steps: 1) bacterial sample collection, 2) DNA isolation or extraction, 3) DNA library preparation, 4) DNA library sequencing, and 5) data analysis.

Human gut bacterial DNA extraction is a tedious and very unpleasant job because it often starts off with collecting, weighing, and homogenizing a sample containing human feces. For example, FIG. 1 shows certain initial steps of a conventional method of preparing DNA libraries from a stool sample. These initial steps are often laborious and time-consuming and include stool sample collection, sample weighing, sample homogenization, and extracting the DNA from the homogenized sample through enzymatic digestion, centrifugation, and numerous washing steps. Such conventional methods also require multiple reagents and buffers and expensive one-time-use spin columns. Such conventional methods are also susceptible to high risks of clinician error.

Therefore, a solution is needed which reduces the number of initial operational steps needed to prepare DNA libraries (e.g., 16S metagenomics DNA libraries) from a stool sample for next generation sequencing yet maintain or improve the quantity and quality of target sequence yields compared to conventional methods. Such a solution should be cost-effective compared to conventional methods, require less time, and should lessen the risk of clinician or operator error.

### SUMMARY

Disclosed herein are methods, compositions, and kits for the preparation of DNA libraries directly from a stool sample for downstream next-generation sequencing. In one embodiment, a method comprises applying a stool sample directly to a buffer solution and heating and cooling the buffer solution containing the stool sample. In some embodiments, applying the stool sample directly to the buffer solution can further comprise applying between about 3 mg to 10 mg of the stool sample to about 100 μL of the buffer solution

In certain embodiments, the buffer solution can comprise Tris-HCl, EDTA, and polyacrylic acid. Moreover, heating and cooling the buffer solution can comprise heating the buffer solution containing the stool sample above a temperature threshold and subsequently cooling the buffer solution containing the stool sample to room temperature.

The method can further comprise separating a supernatant within the buffer solution containing the stool sample from a precipitate using centrifugation. The method can also comprise transferring an aliquot of the supernatant into a first reaction vessel containing a first reagent mixture to yield a first reaction mixture. Transferring the aliquot of the supernatant into the first reagent mixture can further comprise transferring about 2 μL of the supernatant into the first reagent mixture in the first reaction vessel.

In some embodiments, the first reagent mixture can comprise Taq DNA polymerase, dNTPs, a primer pool comprising a plurality of forward primers and reverse primers, a cofactor, a nonionic surfactant, a gelatin solution, a glycerol solution, and a reagent buffer.

In some embodiments, the primer pool can comprise a plurality of 16S forward primers and 16S reverse primers for targeting variable regions V3 and V4 of the 16S ribosomal ribonucleic acid (rRNA) gene. Moreover, the reagent buffer can comprise a Tris-HCl buffer solution and a potassium chloride (KCl) buffer solution. In addition, the cofactor can be magnesium chloride (MgCl₂) and the nonionic surfactant can be a polysorbate 20 solution

The method can also comprise subjecting the first reaction mixture in the first reaction vessel to a first polymerase chain reaction (PCR) protocol. The first PCR protocol can comprise the steps of: (i) heating the first reaction mixture at a first temperature to activate the Taq DNA polymerase in an activation step, (ii) further heating the first reaction mixture at a second temperature to denature nucleic acids within the first reaction mixture, (iii) lowering the temperature to a third temperature to allow for annealing and extension, (iv) repeating steps (ii) and (iii) for at least 4 more cycles, (v) further heating the first reaction mixture at a fourth temperature to further denature nucleic acids within the first reaction mixture, (vi) lowering the temperature to a fifth temperature to allow for annealing and extension, (vii) repeating steps (v) and (vi) for at least 24 more cycles, and (viii) holding the amplified first reaction mixture within the first reaction vessel at a holding temperature.

The method can also comprise purifying the first reaction mixture within the first reaction vessel through a first purification procedure. The first purification procedure can comprise: (a) introducing a magnetic bead suspension to the first reaction vessel, (b) incubating a mixture within the first reaction vessel comprising the magnetic bead suspension at room temperature for an incubation period to allow amplicons to bind to beads within the magnetic bead suspension, (c) collecting and immobilizing the amplicon-bound magnetic beads to at least one inner surface of the first reaction vessel by placing at least one outer surface of the first reaction vessel in proximity to a magnet, (d) removing and discarding a supernatant from the first reaction vessel while the amplicon-bound magnetic beads are immobilized to the at least one inner surface of the first reaction vessel by the magnet, (e) introducing an ethanol wash solution to the first reaction vessel comprising the amplicon-bound magnetic beads, (f) removing and discarding a supernatant from the first reaction vessel while the amplicon-bound magnetic beads are immobilized to the at least one inner surface of the first reaction vessel by the magnet, (g) introducing water to the first reaction vessel to elute amplicons bound to the magnetic beads, (h) removing a first amplicon-containing supernatant from the first reaction vessel after the introduction of water while the magnetic beads are immobilized to the at least one inner surface of the first reaction vessel by the magnet, (i) adding the first amplicon-containing supernatant from step (h) to an intermediary reaction vessel and repeating steps (a) through (g) using contents within the intermediary reaction vessel, and (j) removing a second amplicon-containing supernatant from the intermediary reaction vessel after the introduction of the water while the magnetic beads are immobilized to at least one inner surface of the intermediary reaction vessel by the magnet. The second amplicon-containing supernatant removed is a purified target amplicon solution that can be further amplified and purified through subsequent steps of the method.

In some embodiments, the first reaction vessel can be a standalone reaction tube or container such as a standalone PCR tube. In other embodiments, the reaction vessel can be a well of a multi-well plate. The magnet can be part of a magnetic separation rack or platform.

In certain embodiments, collecting and immobilizing the amplicon-bound magnetic beads can further comprise positioning the at least one outer surface of the first reaction vessel next to the magnet and repeatedly moving the first reaction vessel away from the magnet and back next to the magnet.

The method can further comprise transferring the purified target amplicon solution to a second reaction vessel comprising a second reagent mixture to yield a second reaction mixture.

In some embodiments, the second reagent mixture can comprise Taq DNA polymerase, dNTPs, a plurality of sequencing index adapters, a cofactor, a nonionic surfactant, a gelatin solution, a glycerol solution, and a reagent buffer.

The method can further comprise subjecting the second reaction mixture in the second reaction vessel to a second PCR protocol. The second PCR protocol can comprise the steps of: (i) heating the second reaction mixture at a first temperature to activate the Taq DNA polymerase in an activation step; (ii) further heating the second reaction mixture at a second temperature to denature nucleic acids within the reaction mixture; (iii) lowering the temperature to a third temperature to allow for annealing and extension, (iv) adjusting the temperature to a fourth temperature to allow for additional extension, (v) repeating steps (ii) through (iv) for at least 7 more cycles, (vi) further heating the second reaction mixture at a fifth temperature to allow for further extension, and (vii) holding the amplified second reaction mixture within the second reaction vessel at a holding temperature.

The method can further comprise purifying index-tagged amplicons within the second reaction vessel through a second purification procedure to yield a purified index-tagged DNA library. The second purification procedure can comprise (a) introducing additional instances of the magnetic bead suspension to the second reaction vessel, (b) incubating a mixture within the second reaction vessel comprising the magnetic bead suspension at room temperature for an incubation period to allow amplicons to bind to beads within the magnetic bead suspension, (c) collecting and immobilizing the amplicon-bound magnetic beads to at least one inner surface of the second reaction vessel by placing at least one outer surface of the second reaction vessel in proximity to a magnet, (d) removing and discarding a supernatant from the second reaction vessel while the amplicon-bound magnetic beads are immobilized to the at least one inner surface of the second reaction vessel by the magnet, (e) introducing an ethanol wash solution to the second reaction vessel comprising the amplicon-bound magnetic beads, (f) removing and discarding a supernatant from the second reaction vessel while the amplicon-bound magnetic beads are immobilized to the at least one inner surface of the second reaction vessel by the magnet, (g) introducing water to the second reaction vessel to elute amplicons bound to the magnetic beads, (h) removing a first amplicon-containing supernatant from the second reaction vessel after the introduction of the water while the magnetic beads are immobilized to the at least one inner surface of the second reaction vessel by the magnet, (i) adding the first amplicon-containing supernatant from step (h) to another intermediary reaction vessel and repeating steps (a) through (g) using contents within the other intermediary reaction vessel, and (j) removing a second amplicon-containing supernatant from the other intermediary reaction vessel after the introduction of the water while the magnetic beads are immobilized to at least one inner surface of the other intermediary reaction vessel by the magnet.

The second amplicon-containing supernatant removed is the purified DNA library that can be sequenced using a next-generation sequencing protocol. For example, the DNA library generated from this method can be sequenced using an Illumina® NGS protocol, an Ion PGM® protocol, a SOLiD® NGS protocol, or a combination thereof.

### BRIEF DESCRIPTION OF THE DRAWINGS

FIG. 1 illustrates certain steps of a method known in the art for preparing DNA libraries from a stool sample.

FIG. 2 illustrates an embodiment of an improved method of preparing a DNA library directly from a stool sample.

FIG. 3 illustrates the introduction of a stool sample into a buffer solution using a swab.

FIG. 4 illustrates certain initial steps of the method for preparing a DNA library directly from a stool sample.

FIG. 5 illustrates an embodiment of a first purification procedure using magnetic beads.

FIG. 6A illustrates an embodiment of a magnetic separation rack used as part of a purification procedure.

FIG. 6B illustrates another embodiment of a magnetic separation rack used as part of the purification procedure.

FIG. 6C illustrates an embodiment of a multi-well plate positioned on the magnetic separation rack shown in FIG. 6B.

FIG. 6D is a black-and-white image illustrating an embodiment of a multi-well plate having magnetic beads immobilized to an inner side surface of wells of the well plate.

FIG. 7 illustrates additional steps of the method for preparing a DNA library directly from a stool sample.

FIG. 8 illustrates an embodiment of a second purification procedure using magnetic beads.

FIG. 9 is bioanalyzer trace showing the size distribution of a 16S DNA library prepared directly from a stool sample using the method disclosed herein.

FIGS. 10A to 10C illustrate comparisons of 16S DNA libraries prepared from three different stool samples using traditional DNA extraction methods and 16S DNA libraries prepared from the same three stool samples using the method disclosed herein

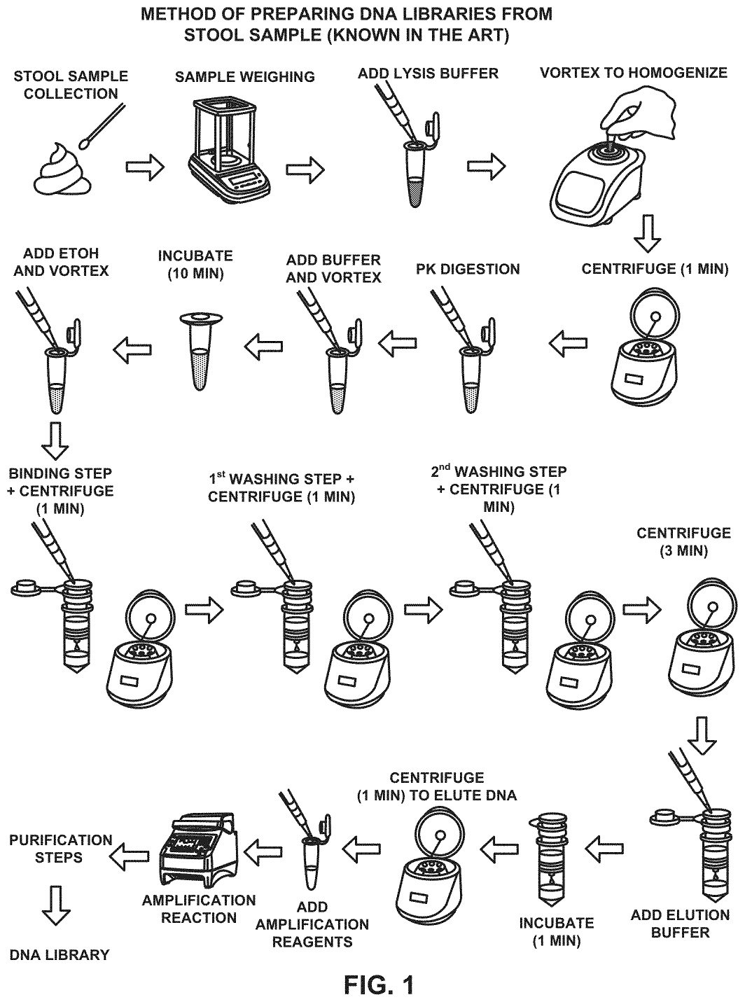

### DETAILED DESCRIPTION

Disclosed herein are methods, compositions, and kits for the preparation of DNA libraries directly from a stool sample for metagenomics next generation sequencing. A DNA library is a collection of genomic DNA sequences of interest obtained from a tissue sample of an organism. The methods, compositions, and kits disclosed herein are optimized for the preparation of DNA libraries for further downstream next-generation sequencing (NGS) for clinical diagnosis and research. For example, the DNA libraries prepared using the methods, compositions, and kits disclosed herein can be used to identify or diagnose pathogenic bacteria. The DNA libraries generated from the methods, compositions, and kits disclosed herein can be used with any number of NGS platforms, including platforms requiring immobilization of DNA fragments onto a solid support, cyclic sequencing reactions using automated devices, and detection of sequences using imaging or semiconductor technologies. For example, the DNA libraries generated from the methods, compositions, and kits disclosed herein can be used with an Illumina® sequencing by synthesis (SBS) NGS platform (e.g., an Illumina MiniSeq®, MiSeq®, or NextSeq® system) distributed by Illumina, Inc., an Ion Personal Genome Machine® (PGM) system distributed by Thermo Fisher Scientific Inc., a SOLiD® NGS system distributed by Thermo Fisher Scientific Inc., or a combination thereof.

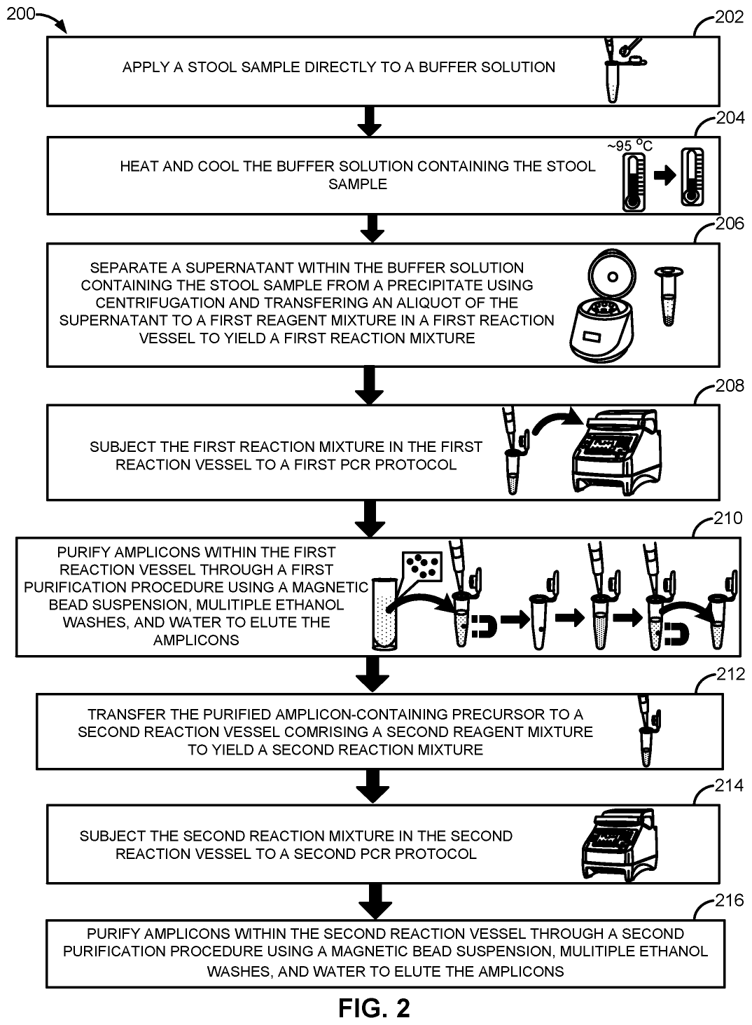

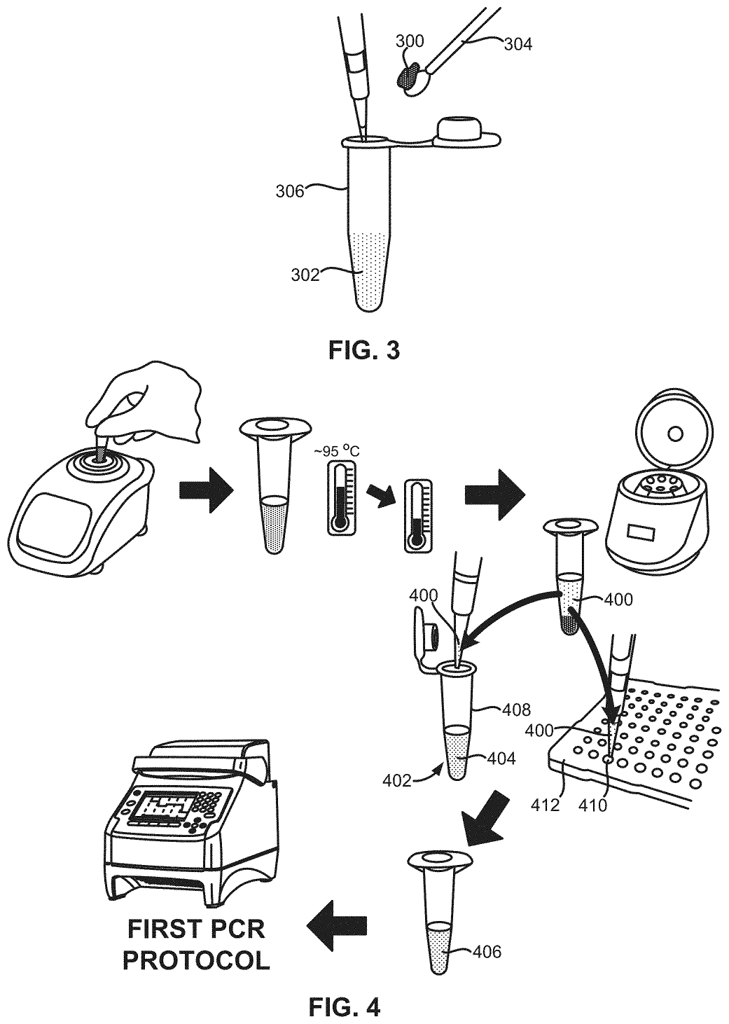

FIG. 2 illustrates an embodiment of an improved method 200 of preparing a DNA library directly from a stool sample 300 (see FIG. 3). The stool sample 300 can be a fecal sample obtained from a human subject (i.e., human fecal matter) or an animal subject (i.e., animal fecal matter). In some embodiments, the stool sample 300 can comprise trace amounts of contaminants such as dirt, mud, hair, or other environmental contaminants.

The method 200 can also be used to prepare a DNA library directly from a sample of vomit. In further embodiments, the method 200 can also be used to prepare a DNA library directly from an environmental sample such as a sample of mud or a slurry comprising sand and liquid.

The method 200 can comprise applying the stool sample 300 to a buffer solution 302 (see FIG. 3) in operation 202. For example, the stool sample 300 can be dropped or stirred into the buffer solution 302. In other embodiments, the stool sample 300 can be smeared along an inner surface of a sample collection tube containing the buffer solution 302.

Applying the stool sample 300 directly to the buffer solution 302 further comprises applying between about 3 mg to about 10 mg of the stool sample 300 to between about 100 μL to about 150 μL of the buffer solution 302. In other embodiments, applying the stool sample 300 directly to the buffer solution 302 can comprise applying about 5 mg of the stool sample 300 to about 100 μL of the buffer solution 302.

The buffer solution 302 can comprise a tris(hydroxymethyl)aminomethane-hydrochloric acid (Tris-HCl) buffer, ethylenediaminetetraacetic acid (EDTA), and polyacrylic acid.

Presented in Table 1 below is an example formulation of the buffer solution 302:

TABLE 1
EXAMPLE BUFFER SOLUTION

| Solution Component | Concentration | |
|---|---|---|
| Tris-HCl, pH 8.0 | 30.0.0 | mM |
| EDTA | 5 | mM |

| | |
|---|---|
| Polyacrylic acid | 0.10% (v/v) |

The method 200 can further comprise heating and cooling the buffer solution 302 containing the stool sample 300 in operation 204. Operation 204 can also comprise homogenizing the buffer solution 302 prior to heating and cooling the buffer solution 302 containing the stool sample 300. For example, the method 200 can also comprise homogenizing the buffer solution 302 containing the stool sample 300 using a vortex mixer or shaker. In other embodiments, the stool sample 300 can be stirred into the buffer solution 302 using a stirring rod or stirrer.

The method 200 can further comprise heating the buffer solution 302 containing the stool sample 300 above a threshold temperature and subsequently cooling the buffer solution 302 containing the stool sample 300 to room temperature in operation 204. In some embodiments, the threshold temperature can be between about 90° C. to about 100° C. More specifically, the threshold temperature can be about 95° C.

One unexpected discovery made by the applicant is that the buffer solution 302 having the composition disclosed herein combined with the heating and cooling steps disclosed herein are effective in facilitating the breakdown of the stool sample 300 without unduly interfering with the quality of the nucleic acids for further downstream processing.

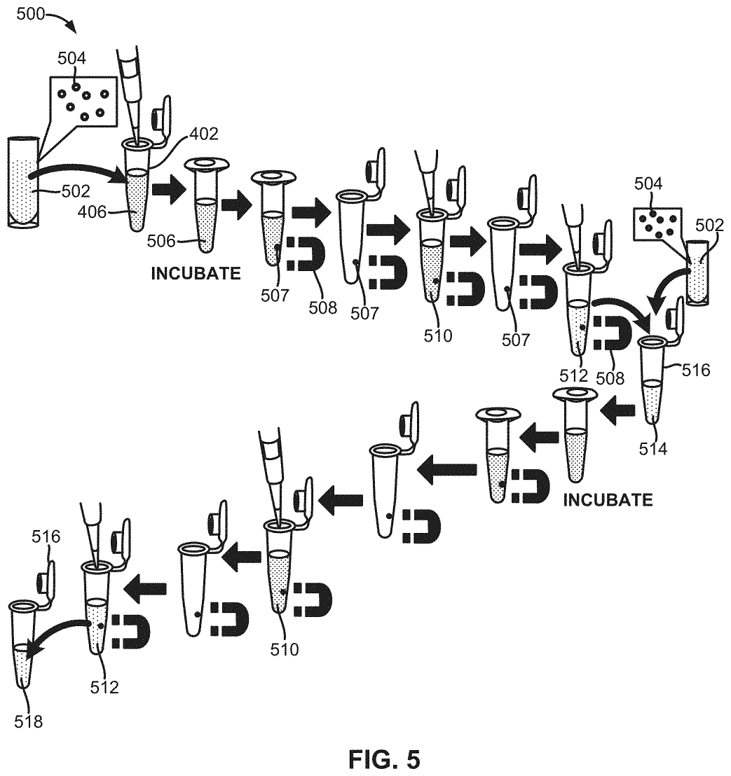

The method 200 can also comprise separating a supernatant 400 (see FIG. 4) within the buffer solution 302 containing the stool sample 300 from a precipitate using centrifugation in operation 206. The buffer solution 302 containing the stool sample 300 can be centrifuged when the solution has reached room temperature. Operation 206 can also comprise transferring an aliquot of the supernatant 400 into a first reaction vessel 402 containing a first reagent mixture 404 to yield a first reaction mixture 406 (see, FIG. 4).

In some embodiments, transferring the aliquot of the supernatant 400 into the first reaction vessel 402 containing the first reagent mixture 404 can comprise transferring about 2 μL of the supernatant 400 into the first reaction vessel 402 containing about 18 μL of the first reagent mixture 404. In other embodiments, transferring the aliquot of the supernatant 400 into the first reaction vessel 402 containing the first reagent mixture 404 can comprise transferring between about 2 μL to about 5 μL of the supernatant 400 into the first reaction vessel 402 containing the first reagent mixture 404. The aliquot of the supernatant 400 can be transferred using a pipette such as a fixed-volume micropipette or an adjustable-volume micropipette.

The first reagent mixture 404 can comprise a reagent solution and a primer pool comprising a plurality of forward primers and reverse primers for the target sequences of interest. The reagent solution can comprise a DNA polymerase, a plurality of dNTPs, a cofactor, a nonionic surfactant, a gelatin solution, a glycerol solution, and one or more reagent buffers.

In some embodiments, the one or more reagent buffers can comprise a tris(hydroxymethyl)aminomethane (Tris) buffer solution (e.g., a Tris-hydrochloric acid (HCl) buffer solution), a potassium chloride (KCl) buffer solution, or a combination thereof.

In some embodiments, the cofactor or cofactor solution can be a magnesium chloride (MgCl₂) solution. The nonionic surfactant can be a polysorbate solution (e.g., a polysorbate 20 solution). More specifically, the nonionic surfactant can be a Tween® 20 surfactant distributed by Sigma-Aldrich, Inc., a Montanox™ 20 surfactant distributed by SEPPIC S.A., or an Alkest® 20 surfactant distributed by Oxiteno S.A.

In some embodiments, the DNA polymerase can be a thermostable DNA polymerase such as a Taq DNA polymerase. For example, the Taq DNA polymerase can be a Taq DNA polymerase provided by Thermo Fisher Scientific Inc.

The concentration of the Tris-HCl buffer solution can be between about 60.0 mM and about 90.0 mM. The pH of the Tris-HCl buffer can be at a pH of about 8.0. The concentration of the KCl buffer solution can be between about 100.0 mM and about 150.0 mM. The concentration of the MgCl₂ solution can be between about 2.0 mM and about 5.0 mM. The polysorbate 20 solution can be between about 0.01% (v/v) and about 0.30% (v/v) of the total volume of the reagent solution. The glycerol solution can be between about 10.0% (v/v) and about 30.0% (v/v) of the total volume of the reagent solution. The gelatin solution can be between about 0.01% (v/v) and about 0.80% (v/v) of the total volume of the reagent solution.

As contemplated by this disclosure and as will be appreciated by one of ordinary skill in the art, the reagent solution can be made at different concentrations and provided as 1× to 5× (e.g., 1×, 2×, 3×, 4×, or 5×) master mixes. Presented in Table 2 below is an example formulation of a reagent solution:

TABLE 2
EXAMPLE COMPOSITION OF 2X REAGENT SOLUTION

| Solution Component | Concentration |
|---|---|
| Taq DNA polymerase | 0.5 Units/μL-0.8 Units/μL |
| dNTPs (dATP, dCTP, dGTP, and dTTP) | 1.0 mM-3.0 mM |
| MgCl₂ | 2.0 mM-5.0 mM |
| Polysorbate 20 | 0.01%-0.30% (v/v) |
| Glycerol | 10.0%-30.0% (v/v) |
| Gelatin | 0.01%-0.80% (v/v) |
| KCl | 100.0 mM-150.0 mM |
| Tris-HCl, pH 8.0 | 60.0 mM-90.0 mM |

In some embodiments, 90% (v/v) of the first reaction mixture 406 can be the first reagent mixture 404 and 10% (v/v) of the first reaction mixture 406 can be the supernatant 400. More specifically, the first reaction mixture 406 can be comprised of 50% (v/v) 2× reagent solution, 20% (v/v) 20× primer pool solution, 20% (v/v) deionized water 410, and 10% (v/v) supernatant 400. Presented in Table 3 below is an example formulation of 20 μL of the first reaction mixture 406:

TABLE 3
EXAMPLE FIRST REACTION MIXTURE COMPOSITION

| | | Percentage of |
|---|---|---|
| Droplet Component | Volume | Total Volume |
| 2X Reagent Solution | 10 μL | 50% |
| 20X 16S Primer Pool | 4 μL | 20% |
| Deionized water | 4 μL | 20% |
| Supernatant containing nucleic acids | 2 μL | 10% |
| TOTAL: | 20 μL | 100% |

In other embodiments, the first reaction mixture 406 can be comprised of 50% (v/v) 2× reagent solution, 5% (v/v) 20× primer pool solution, 35% (v/v) deionized water 410, and 10% (v/v) supernatant 400. Presented in Table 4 below is another example formulation of 20 μL of the first reaction mixture 406:

TABLE 4
EXAMPLE FIRST REACTION MIXTURE COMPOSITION

| | | Percentage of |
|---|---|---|
| Droplet Component | Volume | Total Volume |
| 2X Reagent Solution | 10 μL | 50% |
| 20X 16S Primer Pool | 1 μL | 5% |
| Deionized water | 7 μL | 35% |
| Supernatant containing nucleic acids | 2 μL | 10% |
| TOTAL: | 20 μL | 100% |

In some embodiments, the primer pool can comprise a plurality of forward primers and reverse primers. For example, the primer pool can comprise a plurality of 16S forward primers and 16S reverse primers for targeting variable regions V3 and V4 of the 16S ribosomal ribonucleic acid (rRNA) gene. When such 16S primers are used, the DNA library generated can be considered a 16S DNA library. In other embodiments, the primer pool can comprise a plurality of forward primers and reverse primers targeting other regions of interest.

The method 200 can further comprise subjecting the first reaction mixture 406 in the first reaction vessel 402 to a first polymerase chain reaction (PCR) protocol in step 208. The first PCR protocol can comprise (i) heating the first reaction mixture 406 at a first temperature to activate the DNA polymerase in an activation step. The first PCR protocol can also comprise (ii) further heating the first reaction mixture 406 at a second temperature to denature nucleic acids (template DNA) within the first reaction mixture 406 in a denaturation step. The first PCR protocol can further comprise (iii) lowering the temperature to a third temperature to allow for annealing of the primers to the template DNA and extension or elongation of the annealed primers by the DNA polymerase. The first PCR protocol can also comprise (iv) repeating the (ii) denaturation and (iii) annealing and extension steps for at least 4 more cycles (so 5 cycles total). The first PCR protocol can also comprise (v) further heating the first reaction mixture at a fourth temperature to further denature nucleic acids within the first reaction mixture. The first PCR protocol can further comprise (vi) lowering the temperature to a fifth temperature to allow for further annealing and extension. The first PCR protocol can also comprise (vii) repeating the (v) denaturation and (vi) annealing and extension steps for at least 24 more cycles (so 25 cycles total).

In other embodiments, the (v) denaturation and (vi) annealing and extension steps can be repeated for between 25 cycles and 30 cycles. The first PCR protocol can also comprise holding the amplified first reaction mixture 406 within the first reaction vessel 402 at a holding temperature.

In some embodiments, the first temperature of the first PCR protocol can be about 95° C. (i.e., the activation temperature can be about 95° C.), the second temperature of the first PCR protocol can also be about 95° C. (i.e., the denaturation temperature can be about 95° C.), the third temperature of the first PCR protocol can be about 60° C. (i.e., the annealing and extension temperature can be about 60° C.), the fourth temperature of the first PCR protocol can be about 95° C. (i.e., the denaturation temperature can be about 95° C.), the fifth temperature of the first PCR protocol can be about 72° C. (i.e., the annealing and extension temperature can be about 72° C.), and the holding temperature can be about 8° C.

Presented in Table 5 below is an example first PCR protocol:

TABLE 5
EXAMPLE FIRST PCR PROTOCOL

| Enzyme | | Annealing and | | Annealing and | |
|---|---|---|---|---|---|
| Activation | Denaturation | Extension | Denaturation | Extension | Cooling |
| Step | Step | Steps | Step | Steps | Step |
| Temp: ~95° C. | Temp: ~95° C. | Temp: ~60° C. | Temp: ~95° C. | Temp: ~72° C. | Temp: ~8° C. |
| Time: ~15 min. | Time: ~1 min. | Time: ~6 min. | Time: ~30 sec. | Time: ~3 min. | Hold |

| | |
|---|---|
| 5 Cycles | 25 Cycles |

After undergoing the aforementioned first PCR protocol, the first reaction mixture 406 can be purified to obtain or collect the amplified sequences.

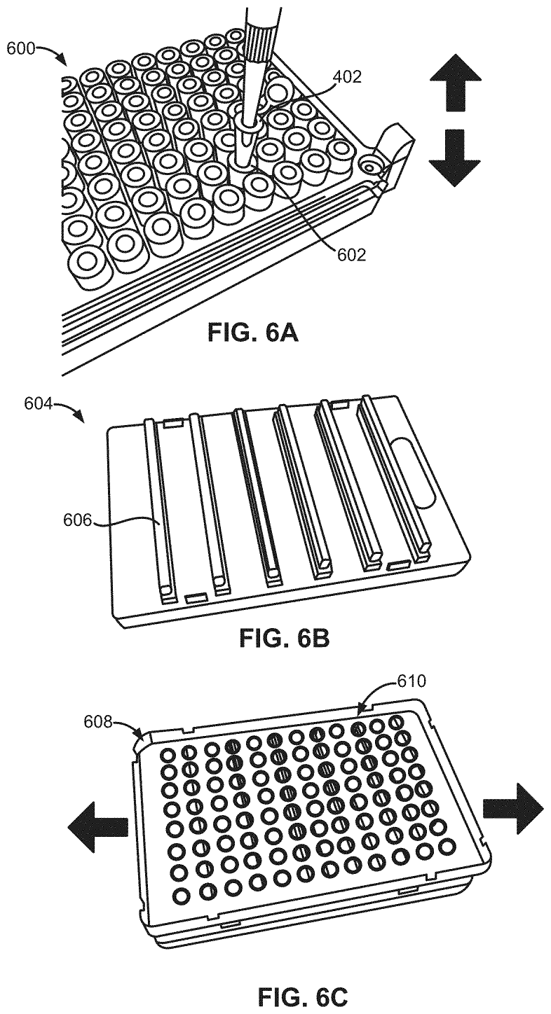

The method 200 can further comprise purifying or isolating the amplicons within the first reaction vessel 402 through a first purification procedure 500 (see, for example, FIG. 5) in operation 210. The first purification procedure 500 can comprise purifying or isolating the amplicons using magnetic beads 504 (see, for example, FIG. 5). The first purification procedure 500 can further comprise subjecting the amplicon-bound magnetic beads 507 to multiple ethanol washes and eluting the target amplicons using water to yield a purified target amplicon solution 518. The first purification procedure 500 will be discussed in more detail in the following sections.

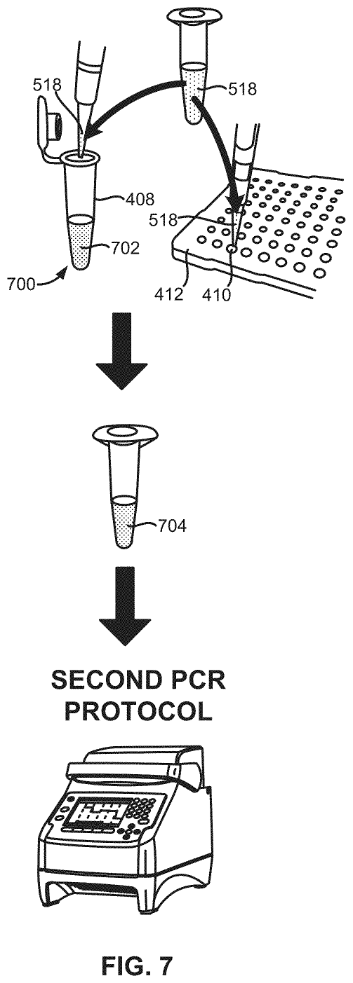

The method 200 can further comprise transferring the purified target amplicon solution 518 to a second reaction vessel 700 comprising a second reagent mixture 702 to yield a second reaction mixture 704 (see, for example, FIG. 7) in operation 212.

In some embodiments, transferring the aliquot of the purified target amplicon solution 518 into the second reaction vessel 700 containing the second reagent mixture 702 can comprise transferring about 10.5 μL of the purified target amplicon solution 518 into the second reaction vessel 700 containing about 14.5 μL of the second reagent mixture 702. The aliquot of the purified target amplicon solution 518 can be transferred using a pipette such as a fixed-volume micropipette or an adjustable-volume micropipette.

The second reagent mixture 702 can comprise a reagent solution and an index primer pool comprising a plurality of index adapter oligonucleotides or index adapters. The reagent solution can be the same reagent solution disclosed in the previous sections (see, e.g., Table 2). For example, the reagent solution can comprise a Taq DNA polymerase, a plurality of dNTPs, a cofactor, a nonionic surfactant, a gelatin solution, a glycerol solution, and one or more reagent buffers.

In some embodiments, about 58% (v/v) of the second reaction mixture 704 can be the second reagent mixture 702 and about 42% (v/v) of the second reaction mixture 704 can be the purified target amplicon solution 518. More specifically, the second reaction mixture 704 can be comprised of 50% (v/v) 2× reagent solution, 8% (v/v) index primer pool solution, and 42% (v/v) purified target amplicon solution 518. Presented in Table 6 below is an example formulation of 25 μL of the second reaction mixture 704:

TABLE 6
EXAMPLE SECOND REACTION MIXTURE COMPOSITION

| | | |
|---|---|---|
| | | Percentage of |
| Droplet Component | Volume | Total Volume |

| | | | |
|---|---|---|---|
| 2X Reagent Solution | 12.5 | μL | 50% |
| Nextera ® XT Index 1 Primers | 1 | μL | 4% |
| Nextera ® XT Index 2 Primers | 1 | μL | 4% |
| Purified target amplicon solution | 10.5 | μL | 42% |
| TOTAL: | 25 | μL | 100% |

Presented in Table 7 below is another example formulation of 12.5 μL of the second reaction mixture 704:

TABLE 7
EXAMPLE SECOND REACTION MIXTURE COMPOSITION

| | | |
|---|---|---|
| | | Percentage of |
| Droplet Component | Volume | Total Volume |

| | | | |
|---|---|---|---|
| 2X Reagent Solution | 6.25 | μL | 50% |
| Nextera ® XT Index 1 Primers | 0.5 | μL | 4% |
| Nextera ® XT Index 2 Primers | 0.5 | μL | 4% |
| Purified target amplicon solution | 5.25 | μL | 42% |
| TOTAL: | 12.5 | μL | 100% |

In some embodiments, the index primer pool can comprise a plurality of forward index primers and reverse index primers. For example, the index primer pool can comprise a plurality of forward index adapter oligonucleotides or forward index adapters and a plurality reverse index adapter oligonucleotides or reverse index adapters. The index adapters can be annealed or added to the ends of the amplified target sequences (or target amplicons) after the completion of the second PCR protocol. The index adapters when added to the ends of the target amplicons can act as barcodes or unique identifiers to identify the target amplicons when the DNA library is being sequenced using next-generation sequencing. Once the target amplicons are tagged with the index adapters, the DNA library can be considered ready for sequencing using next-generation sequencing systems such as the Illumina MiSeq® system. Different pairs of index adapters can also be used to allow multiple pooled samples to be sequenced together in a single high-throughput next-generation sequencing run.

The method 200 can further comprise subjecting the second reaction mixture 702 in the second reaction vessel 700 to a second PCR protocol in operation 214. The second PCR protocol can be a limited-cycle protocol for adding index adapters to the ends of the target amplicons (i.e., index-tagging the target amplicons) and amplifying the index-tagged target amplicons. For example, when the sequence of interest is the 16S rRNA gene, the second PCR protocol can be a limited-cycle protocol for index-tagging the 16S amplicons and amplifying the index-tagged 16S amplicons.

The second PCR protocol can comprise (i) heating the second reaction mixture 702 at a first temperature to activate the DNA polymerase in an activation step. The second PCR protocol can also comprise (ii) further heating the second reaction mixture 702 at a second temperature to denature nucleic acids within the second reaction mixture 702 in a denaturation step.

The second PCR protocol can further comprise (iii) lowering the temperature to a third temperature to allow for annealing of the index primers to the target amplicons and extension or elongation of the annealed index primers by the DNA polymerase. The second PCR protocol can also comprise (iv) further heating at a fourth temperature to allow for further extension. The second PCR protocol can also comprise (v) repeating the (ii) denaturation, (iii) annealing and extension, and (iv) further extension steps for at least 7 more cycles (so 8 cycles total). In other embodiments, steps (ii) through (v) can be repeated for between 8 cycles and 10 cycles.

The second PCR protocol can also comprise (vi) further heating the second reaction mixture 702 at a fifth temperature to allow for further extension. The second PCR protocol can also comprise holding the index-tagged amplicons within the second reaction vessel 700 at a holding temperature.

In some embodiments, the first temperature of the second PCR protocol can be about 95° C. (i.e., the activation temperature can be about 95° C.), the second temperature of the second PCR protocol can also be about 95° C. (i.e., the denaturation temperature can be about 95° C.), the third temperature of the second PCR protocol can be about 66° C. (i.e., the annealing and extension temperature can be about 66° C.), the fourth temperature of the second PCR protocol can be about 72° C. (i.e., the further extension temperature can be about 72° C.), the fifth temperature of the second PCR protocol can be about 72° C. (i.e., the final extension temperature can be about 72° C.), and the holding temperature can be about 4° C.

Presented in Table 8 below is an example second PCR protocol:

TABLE 8
EXAMPLE SECOND PCR PROTOCOL

| Enzyme | | Annealing and | | Final | |
|---|---|---|---|---|---|
| Activation | Denaturation | Extension | Extension | Extension | Cooling |
| Step | Step | Steps | Step | Step | Step |
| Temp: ~95° C. | Temp: ~95° C. | Temp: ~66° C. | Temp: ~72° C. | Temp: ~72° C. | Temp: ~4° C. |
| Time: ~2 min. | Time: ~30 sec. | Time: ~30 sec. | Time: ~60 sec. | Time: ~5 min. | Hold |

8 Cycles

After undergoing the aforementioned second PCR protocol, the index-tagged amplicons can be purified to obtain an index-tagged DNA library ready for next generating sequencing.

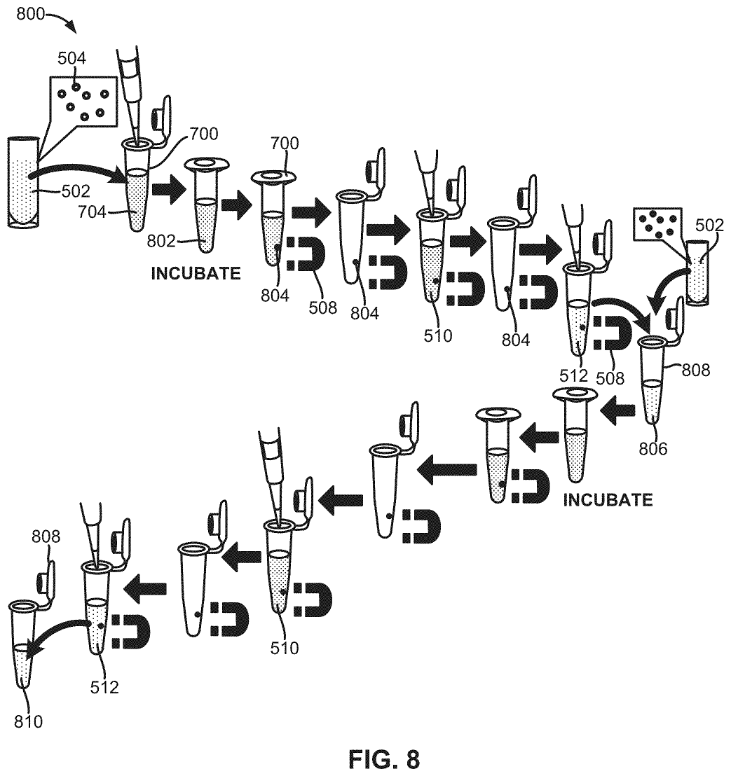

The method 200 can further comprise purifying or isolating the index-tagged amplicons within the second reaction vessel 700 through a second purification procedure 800 (see, for example, FIG. 8) in operation 216. The second purification procedure 800 can comprise purifying or isolating the index-tagged amplicons using magnetic beads 504 (see, for example, FIG. 8). The second purification procedure 800 can further comprise subjecting the amplicon-bound magnetic beads 804 to multiple ethanol washes and eluting the index-tagged amplicons using water to yield a purified index-tagged DNA library 810. The second purification procedure 800 will be discussed in more detail in the following sections.

The index-tagged DNA library 810 generated from this method 200 can be sequenced using (but not limited to) an Illumina® NGS protocol, an Ion PGM® protocol, a SOLiD® NGS protocol, or a combination thereof.

One unexpected discovery made by the applicant is that the first PCR protocol disclosed herein is effective in amplifying target sequences from the buffer solution comprising the stool sample. Moreover, the amplified sequences obtained from the aforementioned first PCR protocol are uniform and high in quantity.

Another unexpected discovery is that the purification procedures disclosed herein (e.g., the first purification procedure 500 and the second purification procedure 800) are effective in purifying the target amplicons after the first PCR protocol and the index-tagged target amplicons after the second PCR protocol. Moreover, the DNA library (e.g., a 16S DNA library) resulting from such purification procedures are of high-quality and ready for sequencing using NGS protocols.

Yet another unexpected discovery made by the applicant is that DNA libraries (e.g., 16S DNA libraries) could be prepared from much smaller amounts of stool sample using the method 200 disclosed herein than traditional extraction methods.

FIG. 3 illustrates that the stool sample 300 can be applied using a swab 302 or pick. In some embodiments, the swab 304 can be a traditional cotton swab or Q-tip. In other embodiments, the swab 304 can comprise a polymeric swab head and handle. For example, the swab 304 can comprise a polyester fabric head and a polypropylene handle. In further embodiments, the swab 304 can comprise a swab head made of Teflon coated fiberglass.

In some embodiments, the swab 302 carrying the stool sample 300 can drop the stool sample 300 into the buffer solution 302. In other embodiments, the stool sample 300 can be stirred into the buffer solution 302 using the swab 302.

FIG. 4 illustrates that the supernatant 400 within the buffer solution 302 containing the stool sample 300 can be separated from a precipitate using centrifugation. The buffer solution 302 containing the stool sample 300 can be centrifuged when the solution reaches room temperature. FIG. 4 also illustrates that an aliquot of the supernatant 400 can be transferred into a first reaction vessel 402 containing a first reagent mixture 404 to yield a first reaction mixture 406.

In some embodiments, the first reaction vessel 402 can be a single PCR reaction tube 408 or vessel. In other embodiments, the first reaction vessel 402 can be one well 410 of a multi-well plate 412 (e.g., a multi-well PCR plate), such as a 96-well plate or a 384-well plate.

In some embodiments, transferring the aliquot of the supernatant 400 into the first reaction vessel 402 containing the first reagent mixture 404 can comprise transferring about 2 μL of the supernatant 400 into the first reaction vessel 402 containing about 18 μL of the first reagent mixture 404. In other embodiments, transferring the aliquot of the supernatant 400 into the first reaction vessel 402 containing the first reagent mixture 404 can comprise transferring between about 2 μL to about 5 μL of the supernatant 400 into the first reaction vessel 402 containing the first reagent mixture 404. The aliquot of the supernatant 400 can be transferred using a pipette such as a fixed-volume micropipette or an adjustable-volume micropipette.

The first reagent mixture 404 can comprise a reagent solution and a primer pool comprising a plurality of forward primers and reverse primers for the target sequences of interest. The reagent solution can comprise a DNA polymerase, a plurality of dNTPs, a cofactor, a nonionic surfactant, a gelatin solution, a glycerol solution, and one or more reagent buffers.

FIG. 5 illustrates an embodiment of the first purification procedure 500. The first purification procedure 500 can comprise introducing a magnetic bead suspension 502 to the first reaction vessel 402. The magnetic bead suspension 502 can comprise magnetic beads 504 configured to allow the target amplicons within the amplified first reaction mixture 406 to selectively bind to surfaces of the magnetic beads 504. For example, the magnetic bead suspension 502 can be AMPure® beads manufactured by Beckman Coulter, Inc.

In some embodiments, the volume of the magnetic bead suspension 502 added is anywhere between 1× to 1.8× the volume of the first reaction mixture 406 within the first reaction vessel 402. For example, 36 μL of the magnetic bead suspension 502 can be added to 20 μL of the first reaction mixture 406 within the first reaction vessel 402.

The first purification procedure 500 can also comprise incubating a mixture 506 within the first reaction vessel 402 comprising both the amplified first reaction mixture 406 and the magnetic bead suspension 502 at room temperature (e.g., between about 20° C. to about 25° C.) for an incubation period (e.g., between about 5 minutes to 10 minutes) to allow the target amplicons to bind to the magnetic beads 504.

The first purification procedure 500 can also comprise collecting and immobilizing amplicon-bound magnetic beads 507 to at least one inner surface of the first reaction vessel 402 by placing at least one outer surface of the first reaction vessel 402 in proximity to a magnet 508. The first purification procedure 500 can further comprise positioning the at least one outer surface of the first reaction vessel 402 in proximity to the magnet 508 and then repeatedly moving the first reaction vessel 402 away from the magnet 508 and bringing the at least one outer surface of the reaction vessel 402 back next to the magnet 508.

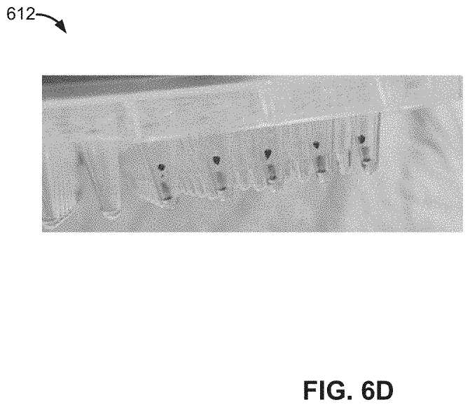

In some embodiments, the magnet 508 can be a permanent magnet. For example, the magnet 508 can be a neodymium iron boron (NdFeB) permanent magnet. The magnet 508 can be incorporated into or embedded within a magnetic separation rack or platform (see, e.g., FIGS. 6A and 6B).

The first purification procedure 500 can also comprise removing and discarding a supernatant from the first reaction vessel 402 while the amplicon-bound magnetic beads 507 are immobilized to the at least one inner surface of the first reaction vessel 402 by the magnet 508. Removing and discarding the supernatant can comprise using a micropipette to aspirate the supernatant from the first reaction vessel 402 into the pipette tip and expelling the supernatant to discard the supernatant.

The first purification procedure 500 can also comprise introducing an ethanol wash solution 510 to the first reaction vessel 402 containing the amplicon-bound magnetic beads 507. For example, the ethanol wash solution 510 can be a 70% (v/v) ethanol or isopropyl alcohol solution. The first purification procedure 500 can comprise introducing between about 50 μL to about 125 μL of the ethanol wash solution 510 to the first reaction vessel 402 containing the amplicon-bound magnetic beads 507. One objective of the ethanol wash step is to remove excess salts from buffers added to the first reaction vessel 402 in previous steps of the method 200.

The first purification procedure 500 can further comprise removing and discarding a supernatant comprising primarily of the ethanol wash solution 510 from the first reaction vessel 402 while the amplicon-bound magnetic beads 507 are immobilized to the at least one inner surface of the first reaction vessel 402 by the magnet 508. The first purification procedure can also comprise drying (e.g., air drying) the first reaction vessel 402 after each ethanol wash to evaporate the ethanol left over. The ethanol wash steps can be repeated one or more times in succession. For example, the ethanol wash steps can be performed twice before moving on to the elution step.

The first purification procedure 500 can also comprise introducing water 512 (e.g., deionized water) to the first reaction vessel 502 to elute amplicons bound to the magnetic beads 504. For example, the purification procedure 500 can comprise introducing about 20 μL of deionized water to the first reaction vessel 502 to elute the amplicons bound to the magnetic beads 504. The first purification procedure 500 can further comprise removing a first amplicon-containing supernatant 514 from the first reaction vessel 402 after the introduction of water 512 while the magnetic beads 504 are immobilized to the at least one inner surface of the first reaction vessel 402 by the magnet 508. For example, the first amplicon-containing supernatant 514 can be aspirated from the first reaction vessel 402 using a micropipette and transferred to an intermediary reaction vessel 516.

The aforementioned purification steps can then be repeated again using contents within the intermediary reaction vessel 516. For example, the first purification procedure 500 can further comprise introducing additional instances of the magnetic bead suspension 502 to the intermediary reaction vessel 516 and incubating a mixture within the intermediary reaction vessel 516 comprising both the first amplicon-containing supernatant 514 and the magnetic bead suspension 502 at room temperature (e.g., between about 20° C. to about 25° C.) for an incubation period (e.g., between about 5 minutes to 10 minutes) to allow the target amplicons to bind to the magnetic beads 504.

The first purification procedure 500 can also comprise collecting and immobilizing the amplicon-bound magnetic beads to at least one inner surface of the intermediary reaction vessel 516 by placing at least one outer surface of the intermediary reaction vessel 516 in proximity to a magnet 508. The first purification procedure 500 can also comprise removing and discarding a supernatant from the intermediary reaction vessel 516 while the amplicon-bound magnetic beads are immobilized to the at least one inner surface of the intermediary reaction vessel 516 by the magnet 508.

The first purification procedure 500 can also comprise introducing an ethanol wash solution 510 to the intermediary reaction vessel 516 containing the amplicon-bound magnetic beads. The first purification procedure 500 can further comprise removing and discarding a supernatant comprising primarily of the ethanol wash solution 510 from the intermediary reaction vessel 516 while the amplicon-bound magnetic beads are immobilized. The first purification procedure can also comprise drying (e.g., air drying) the intermediary reaction vessel 516 after each ethanol wash to evaporate the ethanol left over. The ethanol wash steps can be repeated one or more times in succession. For example, the ethanol wash steps can be performed twice before moving on to the elution step. The first purification procedure 500 can also comprise introducing water 512 (e.g., deionized water) to the intermediary reaction vessel 516 to elute target amplicons bound to the magnetic beads 504.

The first purification procedure 500 can further comprise removing a second amplicon-containing supernatant from the intermediary reaction vessel after the introduction of the water while the magnetic beads 504 are immobilized to at least one inner surface of the intermediary reaction vessel 514 by the magnet 508. The second amplicon-containing supernatant removed from the intermediary reaction vessel is the purified target amplicon solution 518.

Although FIG. 5 illustrates the first reaction vessel 402 and the intermediary reaction vessel 516 as standalone reaction tubes or PCR tubes, it is contemplated by this disclosure that any of the first reaction vessel 402 or the intermediary reaction vessel 516 can be a well of a multi-well plate (e.g., a multi-well PCR plate), such as a 96-well plate or a 384-well plate.

FIG. 6A illustrates an embodiment of a magnetic separation rack 600 comprising a plurality of wells 602 with at least one magnet 508 positioned at the bottom of each well 602. For example, the magnetic separation rack 600 can be a DynaMag® magnetic rack for holding non-skirted or semi-skirted 96-well or 384-well PCR plates. In other embodiments, the magnetic separation rack 600 can be any type of magnetic rack or platform comprising one or more magnets 508 positioned on the bottom or sides of the rack or platform. The magnets 508 of the magnetic separation rack 600 can aggregate and collect the magnetic beads 504 including the amplicon-bound magnetic beads 507.

FIG. 6A illustrates that at least part of a reaction vessel (e.g., the first reaction vessel 402) can be positioned into a well 602 of the magnetic separation rack 600 in proximity to a magnet 508 at the bottom of the well. In some embodiments, the reaction vessel can be lifted out of the well 602 and then placed back into the well 602. This can be repeated until the magnetic beads 504 gather (e.g., as a pellet or in pellet form) at the bottom of the reaction vessel 402. The magnetic beads 504 within the reaction vessel can be immobilized when the reaction vessel 402 is supported upright by the well 602 and at least part of the reaction vessel is positioned within the well 602.

Although FIG. 6A illustrates an instance of the reaction vessel as a singular reaction tube or PCR tube, it is contemplated by this disclosure that the reaction vessel can refer to one well of a multi-well plate (e.g., a well of a 96-well PCR plate) and the entire multi-well plate can be positioned on the magnetic separation rack 600 such that each of the wells of the multi-well plate is positioned within a well 602 of the magnetic separation rack.

FIG. 6B illustrates another embodiment of a magnetic separation rack 604. The magnetic separation rack 604 shown in FIG. 6B can comprise magnets 508 designed as magnetic bar 606 or columnar-type magnets extending (e.g., perpendicularly or angularly) from a bottom surface of the rack. The magnetic separation rack 604 shown in FIG. 6B can be designed for use with skirted multi-well plates (e.g., a skirted 96-well or 384-well PCR plate). For example, the magnetic separation rack 604 can be a DynaMag® side skirted magnetic rack for holding skirted 96-well or skirted 384-well PCR plates.

FIG. 6C illustrates an embodiment of a skirted multi-well plate 608 (e.g., a skirted 96-well or 384-well PCR plate) positioned on the magnetic separation rack 604 of FIG. 6B. At least one column of wells 610 of the multi-well plate 608 can be positioned next to or in proximity to a magnetic bar 606 of the skirted multi-well plate 608. To collect and immobilize the magnetic beads 504 (including the amplicon-bound magnetic beads), the skirted multi-well plate 608 can be shifted laterally left-to-right and vice versa such that the column of wells 610 is brought close to the magnetic bar 606, briefly moved away from the magnetic bar 606, and then brought back next to the magnetic bar 606. This can be repeated until the magnetic beads 504 gather or accumulate (e.g., as a pellet or in pellet form) near an inner side surface of the wells 610.

In this embodiment, the individual wells (or individual reaction vessels) of the multi-well plate 608 can have at least one outer surface of the well positioned next to the magnetic bar 606, briefly moving or shifting the well away from the magnetic bar 606, and then bringing the well back next to the magnetic bar 606. The magnetic beads 504 within the column of wells 610 can be immobilized when the column of wells 610 is positioned next to the magnetic bar 606.

Although FIG. 6C illustrates a skirted multi-well plate, it is contemplated by this disclosure that the magnetic separation rack 604 of FIG. 6B can also be used with non-skirted multi-well plates or semi-skirted multi-well plates. Moreover, although FIG. 6C illustrates a multi-well plate, the reaction vessel can also be a singular reaction tube or PCR tube or a plurality of such tubes held by clamps, robotic arms, or other types of holders (e.g., in a column or row), and the singular reaction tube or the plurality of tubes can be positioned close to a magnetic bar 606 and then repeatedly shifted away from and back toward the magnetic bar 606 until the magnetic beads 504 are collected and immobilized within the singular reaction tube or tubes. The magnetic beads 504 can be collected and immobilized to an inner side surface of the singular reaction tube when an outer side surface of the singular reaction tube is positioned in proximity to or next to the magnetic bar 606.

FIG. 6D is a black-and-white image illustrating an embodiment of a well plate 612 having magnetic beads 504 immobilized to the inner side surfaces of wells of the well plate 612. As shown in FIG. 6D, the well plate 612 can be a semi-skirted well plate such as a semi-skirted 96-well plate The wells of the well plate 612 can serve as reaction vessels (e.g., the first reaction vessel 402) for undergoing certain steps of the first purification procedure 500 using the magnetic beads 504.

The magnetic separation racks 600 shown in FIGS. 6A-6D can be used as part of the first purification procedure 500, a second procedure 800 (see FIG. 8), or a combination thereof. For example, any of the first reaction vessel 402 and the second reaction vessel 700 can be positioned on the magnetic separation rack 600 in order to collect and immobilize the magnetic beads 504 within such reaction vessels.

FIG. 7 illustrates that an aliquot of the purified target amplicon solution 518 can be transferred to a second reaction vessel 700 comprising a second reagent mixture 702 to yield a second reaction mixture 704.

In some embodiments, the second reaction vessel 700 can be a single PCR reaction tube 408 or vessel. In other embodiments, the second reaction vessel 700 can be one well 410 of a multi-well plate 412 (e.g., a multi-well PCR plate), such as a 96-well plate or a 384-well plate.

In some embodiments, transferring the aliquot of the purified target amplicon solution 518 into the second reaction vessel 700 containing the second reagent mixture 702 can comprise transferring about 10.5 μL of the purified target amplicon solution 518 into the second reaction vessel 700 containing about 14.5 μL of the second reagent mixture 702. The aliquot of the purified target amplicon solution 518 can be transferred using a pipette such as a fixed-volume micropipette or an adjustable-volume micropipette.

The second reagent mixture 702 can comprise a reagent solution and an index primer pool comprising a plurality of index adapter oligonucleotides or index adapters. The reagent solution can comprise a Taq DNA polymerase, a plurality of dNTPs, a cofactor, a nonionic surfactant, a gelatin solution, a glycerol solution, and one or more reagent buffers.

The index adapters can be annealed or added to the ends of the amplified target sequences (or target amplicons) within the purified target amplicon solution after the second PCR protocol. The index adapters when added to the ends of the target amplicons can act as barcodes or unique identifiers to identify the target amplicons when the DNA library is being sequenced using next-generation sequencing. Once the target amplicons are tagged with the index adapters, the DNA library can be considered ready for sequencing using next-generation sequencing systems such as the Illumina MiSeq® system. Different pairs of index adapters can also be used to allow multiple pooled samples to be sequenced together in a single high-throughput next-generation sequencing run.

In some embodiments, the index adapters can be overhang adapters. For example, the index adapters can be Nextera® XT index primers provided by Illumina, Inc. and compatible with Illumina's MiSeq® next-sequencing system. As a more specific example, the index adapters can comprise Nextera® XT index 1 primers (with P7 adapters) and Nextera® XT index 2 primers (with P5 adapters).

FIG. 8 illustrates an embodiment of the second purification procedure 800. The second purification procedure 800 can comprise introducing a magnetic bead suspension 502 to the second reaction vessel 700 after the second PCR protocol. The magnetic bead suspension 502 can comprise magnetic beads 504 configured to allow the index-tagged amplicons within the amplified second reaction mixture 704 to selectively bind to surfaces of the magnetic beads 504. For example, the magnetic bead suspension 502 can be AMPure® beads manufactured by Beckman Coulter, Inc.

In some embodiments, the volume of the magnetic bead suspension 502 added is anywhere between 1× to 1.8× the volume of the second reaction mixture 704 within the second reaction vessel 700. For example, 36 μL of the magnetic bead suspension 502 can be added to 20 μL of the second reaction mixture 704 within the second reaction vessel 700.

The second purification procedure 800 can also comprise incubating a mixture 802 within the second reaction vessel 700 comprising both the amplified second reaction mixture 704 and the magnetic bead suspension 502 at room temperature (e.g., between about 20° C. to about 25° C.) for an incubation period (e.g., between about 5 minutes to 10 minutes) to allow the index-tagged target amplicons to bind to the magnetic beads 504.

The second purification procedure 800 can also comprise collecting and immobilizing the amplicon-bound magnetic beads 804 to at least one inner surface of the second reaction vessel 700 by placing at least one outer surface of the second reaction vessel 700 in proximity to a magnet 508. The second purification procedure 800 can comprise initially positioning the at least one outer surface of the second reaction vessel 700 in proximity to the magnet 508 and then repeatedly moving the second reaction vessel 700 away from the magnet 508 and bringing the at least one outer surface of the second reaction vessel 700 back next to the magnet 508.

In some embodiments, the magnet 508 can be a permanent magnet. For example, the magnet 508 can be a neodymium iron boron (NdFeB) permanent magnet. The magnet 508 can be incorporated into or embedded within a magnetic separation rack or platform (see, e.g., FIGS. 6A and 6B).

The second purification procedure 800 can also comprise removing and discarding a supernatant from the second reaction vessel 700 while the amplicon-bound magnetic beads 804 are immobilized to the at least one inner surface of the second reaction vessel 700 by the magnet 508. Removing and discarding the supernatant can comprise using a micropipette to aspirate the supernatant from the second reaction vessel 700 into the pipette tip and expelling the supernatant to discard the supernatant.

The second purification procedure 800 can also comprise introducing an ethanol wash solution 510 to the second reaction vessel 700 containing the amplicon-bound magnetic beads 804. For example, the ethanol wash solution 510 can be a 70% (v/v) ethanol or isopropyl alcohol solution. The second purification procedure 800 can comprise introducing between about 50 μL to about 125 μL of the ethanol wash solution 510 to the second reaction vessel 700 containing the amplicon-bound magnetic beads 507. One objective of the ethanol wash step is to remove excess salts from buffers added to the second reaction vessel 700 in previous steps of the method 200.

The second purification procedure 800 can further comprise removing and discarding a supernatant comprising primarily of the ethanol wash solution 510 from the second reaction vessel 700 while the amplicon-bound magnetic beads 804 are immobilized to the at least one inner surface of the second reaction vessel 700 by the magnet 508. The second purification procedure 800 can also comprise drying (e.g., air drying) the second reaction vessel 700 after each ethanol wash to evaporate the ethanol left over. The ethanol wash steps can be repeated one or more times in succession. For example, the ethanol wash steps can be performed twice before moving on to the elution step.

The second purification procedure 800 can also comprise introducing water 512 (e.g., deionized water) to the second reaction vessel 700 to elute amplicons bound to the magnetic beads 504. For example, the second purification procedure 800 can comprise introducing about 20 μL of deionized water to the second reaction vessel 700 to elute the amplicons bound to the magnetic beads 504. The second purification procedure 800 can further comprise removing a first amplicon-containing supernatant 806 from the second reaction vessel 700 after the introduction of water 512 while the magnetic beads 504 are immobilized to the at least one inner surface of the second reaction vessel 700 by the magnet 508. For example, the first amplicon-containing supernatant 806 can be aspirated from the second reaction vessel 700 using a micropipette and transferred to an intermediary reaction vessel 808.

The aforementioned purification steps can then be repeated again using contents within the intermediary reaction vessel 808. For example, the second purification procedure 800 can further comprise introducing additional instances of the magnetic bead suspension 502 to the intermediary reaction vessel 808 and incubating a mixture within the intermediary reaction vessel 808 comprising both the first amplicon-containing supernatant 806 and the magnetic bead suspension 502 at room temperature (e.g., between about 20° C. to about 25° C.) for an incubation period (e.g., between about 5 minutes to 10 minutes) to allow the index-tagged target amplicons to bind to the magnetic beads 504.

The second purification procedure 800 can also comprise collecting and immobilizing the amplicon-bound magnetic beads to at least one inner surface of the intermediary reaction vessel 808 by placing at least one outer surface of the intermediary reaction vessel 808 in proximity to a magnet 508. The second purification procedure 800 can also comprise removing and discarding a supernatant from the intermediary reaction vessel 808 while the amplicon-bound magnetic beads are immobilized to the at least one inner surface of the intermediary reaction vessel 808 by the magnet 508.

The second purification procedure 800 can also comprise introducing an ethanol wash solution 510 to the intermediary reaction vessel 808 containing the amplicon-bound magnetic beads. The second purification procedure 800 can further comprise removing and discarding a supernatant comprising primarily of the ethanol wash solution 510 from the intermediary reaction vessel 808 while the amplicon-bound magnetic beads are immobilized. The second purification procedure 800 can also comprise drying (e.g., air drying) the intermediary reaction vessel 808 after each ethanol wash to evaporate the ethanol left over. The ethanol wash steps can be repeated one or more times in succession. For example, the ethanol wash steps can be performed twice before moving on to the elution step. The second purification procedure 800 can also comprise introducing water 512 (e.g., deionized water) to the intermediary reaction vessel 808 to elute the index-tagged target amplicons bound to the magnetic beads 504.

The second purification procedure 800 can further comprise removing a second amplicon-containing supernatant from the intermediary reaction vessel after the introduction of the water while the magnetic beads 504 are immobilized to at least one inner surface of the intermediary reaction vessel 514 by the magnet 508. The second amplicon-containing supernatant removed from the intermediary reaction vessel is the purified index-tagged library 810 that can be sequenced using a next-generation sequencing protocol such as an Illumina® NGS protocol, an Ion Personal Genome Machine® (PGM) protocol, a SOLiD® NGS protocol, or a combination thereof.

Although FIG. 8 illustrates the second reaction vessel 700 and the intermediary reaction vessel 808 as standalone reaction tubes or PCR tubes, it is contemplated by this disclosure that any of the second reaction vessel 700 or the intermediary reaction vessel 808 can be a well of a multi-well plate (e.g., a multi-well PCR plate), such as a 96-well plate or a 384-well plate.

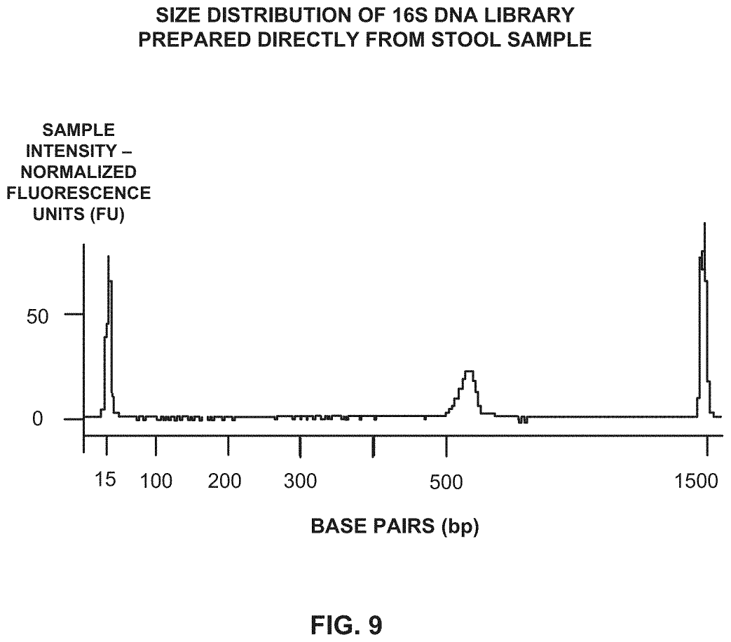

FIG. 9 illustrates the size distribution of a 16S DNA library prepared directly from a stool sample using the method 200 disclosed herein. The 16S DNA library can be analyzed using a bioanalyzer kit or system such as an Agilent® bioanalyzer chip. As previously discussed, the 16S primer pool of the first reaction mixture 406 contained 16S forward and reverse primers targeting the V3 and V4 variable regions of the 16S rRNA gene. Amplicons comprising the V3 and V4 variable regions are expected to have a size of about 500 bp to about 600 bp. As shown in FIG. 9, the 16S DNA library analyzed comprised a significant amount of amplicons of this size when the lower markers (˜15 bp) and upper markers (1500 bp) used for the alignment and quantitation of the DNA library by the bioanalyzer are discounted.

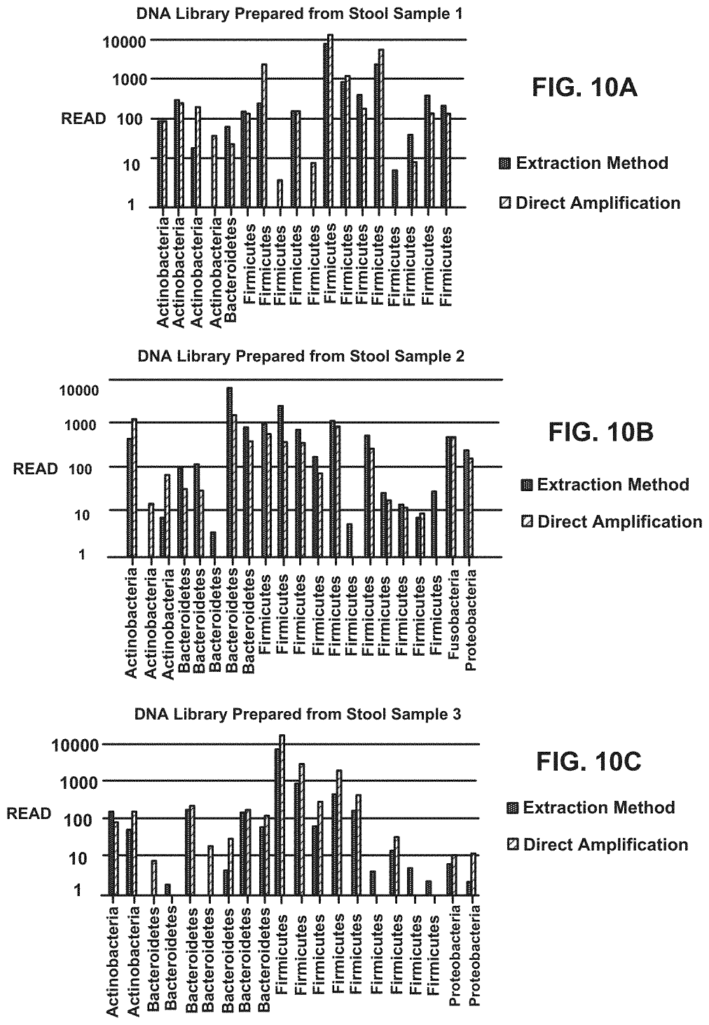

FIGS. 10A to 10C illustrate comparisons of 16S DNA libraries prepared from three different stool samples using traditional DNA extraction methods with a QIAmp® DNA Stool Mini Kit (Cat. No. 51504) and 16S DNA libraries prepared from the same three stool samples using the direct amplification method 200 disclosed herein. As shown in FIGS. 10A to 10C, the method 200 worked at least as well as traditional extraction methods in isolating DNA from bacteria from different taxonomic groups.

Also, important to note here is that the DNA libraries (e.g., the 16S DNA libraries) prepared using the method 200 disclosed herein were each prepared in a shorter period of time than libraries prepared using traditional DNA extraction methods. Moreover, the DNA libraries prepared using the method 200 disclosed herein did not require the user to purchase expensive extraction kits and one-time use spin columns.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±5%) of the modified term such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A method of preparing a deoxyribonucleic acid (DNA) library from a stool sample for downstream next-generation sequencing, comprising:
applying a stool sample directly to a buffer solution;
heating the buffer solution containing the stool sample to a temperature of 90° C. to 100° C. and cooling the buffer solution containing the stool sample to room temperature of 20° C. to 25° C.;
separating a supernatant within the buffer solution containing the stool sample from a precipitate using centrifugation when the buffer solution containing the stool sample has reached the room temperature of 20° C. to 25° C. and transferring an aliquot of the supernatant into a first reaction vessel containing a first reagent mixture to yield a first reaction mixture,
wherein the first reagent mixture comprises:
Taq DNA polymerase,
dNTPs,
a primer pool comprising a plurality of forward primers and reverse primers,
magnesium chloride (MgCl₂),
a nonionic surfactant,
a gelatin solution,
a glycerol solution, and
a buffer solution;
subjecting the first reaction mixture in the first reaction vessel to a first polymerase chain reaction (PCR) protocol;
purifying the first reaction mixture within the first reaction vessel through a first purification procedure using a magnetic bead suspension, and multiple washes using an ethanol wash solution, and water as an eluent to yield a purified target amplicon solution, wherein the first purification procedure further comprises:
(a) introducing the magnetic bead suspension to the first reaction vessel, wherein magnetic beads within the magnetic bead suspension are configured to allow amplicons within the amplified first reaction mixture to selectively bind to surfaces of the magnetic beads;
(b) incubating a mixture within the first reaction vessel comprising the magnetic bead suspension at 20° C. to 25° C. for an incubation period to allow the amplicons to bind to the magnetic beads within the magnetic bead suspension;
(c) collecting and immobilizing the amplicon-bound magnetic beads to at least one inner surface of the first reaction vessel by placing at least one outer surface of the first reaction vessel in proximity to a magnet, wherein the first reaction vessel is a well of a multi-well plate and the magnet is part of a magnetic separation rack or platform and wherein collecting and immobilizing the amplicon-bound magnetic beads further comprises positioning the at least one outer surface of the first reaction vessel next to the magnet, and wherein the method further comprises an additional step of moving the first reaction vessel away from the magnet and bringing the at least one outer surface of the first reaction vessel back next to the magnet;
(d) removing and discarding a supernatant from the first reaction vessel while the amplicon-bound magnetic beads are immobilized to the at least one inner surface of the first reaction vessel by the magnet;
(e) introducing an ethanol wash solution to the first reaction vessel comprising the amplicon-bound magnetic beads;
(f) removing and discarding a wash supernatant from the first reaction vessel while the amplicon-bound magnetic beads are immobilized to the at least one inner surface of the first reaction vessel by the magnet;
(g) introducing water to the first reaction vessel to elute amplicons bound to the magnetic beads;
(h) removing a first amplicon-containing eluate from the first reaction vessel after the introduction of water while the magnetic beads are immobilized to the at least one inner surface of the first reaction vessel by the magnet;
(i) adding the first amplicon-containing eluate from step (h) to an intermediary reaction vessel and repeating steps (a) through (g) using contents within the intermediary reaction vessel; and
(j) removing a second amplicon-containing eluate from the intermediary reaction vessel after the introduction of the water while the magnetic beads are immobilized to at least one inner surface of the intermediary reaction vessel by the magnet, wherein the second amplicon-containing supernatant removed is the purified target amplicon solution;
transferring the purified target amplicon solution to a second reaction vessel comprising a second reagent mixture to yield a second reaction mixture,
wherein the second reagent mixture comprises:
Taq DNA polymerase,
dNTPs,
a plurality of forward and reverse primers comprising index adapter oligonucleotides,
magnesium chloride (MgCl₂),
a nonionic surfactant,
a gelatin solution,
a glycerol solution, and
a buffer solution;
subjecting the second reaction mixture in the second reaction vessel to a second PCR protocol;
purifying index-tagged amplicons within the second reaction vessel through a second purification procedure using additional instances of the magnetic bead suspension and multiple washes using additional instances of the ethanol wash solution and water as an eluent to yield a purified index-tagged DNA library,
wherein the purified index-tagged DNA library is ready for downstream next-generation sequencing.

2. (canceled)

3. (canceled)

4. (canceled)

5. The method of claim 1, wherein the second purification procedure further comprises:
(a) introducing additional instances of the magnetic bead suspension to the second reaction vessel, wherein the magnetic beads within the magnetic bead suspension are configured to allow amplicons within the amplified second reaction mixture to selectively bind to surfaces of the magnetic beads;
(b) incubating a mixture within the second reaction vessel comprising the magnetic bead suspension at 20° C. to 25° C. for an incubation period to allow amplicons to bind to beads within the magnetic bead suspension;
(c) collecting and immobilizing the amplicon-bound magnetic beads to at least one inner surface of the second reaction vessel by placing at least one outer surface of the second reaction vessel in proximity to a magnet;
(d) removing and discarding a supernatant from the second reaction vessel while the amplicon-bound magnetic beads are immobilized to the at least one inner surface of the second reaction vessel by the magnet;
(e) introducing an ethanol wash solution to the second reaction vessel comprising the amplicon-bound magnetic beads;
(f) removing and discarding a wash supernatant from the second reaction vessel while the amplicon-bound magnetic beads are immobilized to the at least one inner surface of the second reaction vessel by the magnet;
(g) introducing water to the second reaction vessel to elute amplicons bound to the magnetic beads;
(h) removing a first amplicon-containing eluate from the second reaction vessel after the introduction of the water while the magnetic beads are immobilized to the at least one inner surface of the second reaction vessel by the magnet;
(i) adding the first amplicon-containing eluate from step (h) to another intermediary reaction vessel and repeating steps (a) through (g) using contents within the other intermediary reaction vessel; and
(j) removing a second amplicon-containing eluate from the other intermediary reaction vessel after the introduction of the water while the magnetic beads are immobilized to at least one inner surface of the other intermediary reaction vessel by the magnet, wherein the second amplicon-containing supernatant removed is the purified index-tagged DNA library.

6. The method of claim 1, wherein the first PCR protocol comprises the steps of:
(i) heating the first reaction mixture to activate the Taq DNA polymerase in an activation step;
(ii) further heating the first reaction mixture to denature nucleic acids within the first reaction mixture;
(iii) lowering the temperature to allow for annealing and extension,
(iv) repeating steps (ii) and (iii) for at least 4 more cycles,
(v) further heating the first reaction mixture to further denature nucleic acids within the first reaction mixture;
(vi) lowering the temperature to allow for annealing and extension,
(vii) repeating steps (v) and (vi) for at least 24 more cycles, and
(viii) holding the amplified first reaction mixture within the first reaction vessel at a holding temperature.

7. The method of claim 6, wherein the second PCR protocol comprises the steps of:
(i) heating the second reaction mixture to activate the Taq DNA polymerase in an activation step;
(ii) further heating the second reaction mixture to denature nucleic acids within the reaction mixture;
(iii) lowering the temperature to allow for annealing and extension,
(iv) raising the temperature to allow for additional extension,
(v) repeating steps (ii) through (iv) for between 7 cycles and 9 cycles,
(vi) further heating the second reaction mixture to allow for further extension, and
(vii) holding the amplified second reaction mixture within the second reaction vessel at a holding temperature, wherein the amplified second reaction mixture is ready for further purification.

8. The method of claim 1, wherein applying the stool sample directly to the buffer solution further comprises applying between 3 mg to 10 mg of the stool sample to 100 μL of the buffer solution and wherein transferring the aliquot of the supernatant into the first reagent mixture in the first reaction vessel further comprises transferring 2 μL of the supernatant into the first reagent mixture in the first reaction vessel.

9. The method of claim 1, wherein the primer pool comprises a plurality of 16S forward primers and 16S reverse primers for targeting variable regions V3 and V4 of the 16S ribosomal ribonucleic acid (rRNA) gene.

10. The method of claim 1, wherein the first and second reagent mixture further comprise a tris(hydroxymethyl)aminomethane (Tris)-hydrochloric acid (HCl) buffer solution and a potassium chloride (KCl) buffer solution, and wherein the nonionic surfactant is a polysorbate 20 solution.

11.-20. (canceled)
